(19) United States
(12) Patent Application Publication
(10) Pub. No.: US 2011/0183892 A1
(43) Pub. Date: Jul. 28, 2011

(54) METHODS FOR THE TREATMENT OF VIRAL CONDITIONS

(76) Inventors: Jonathan D. Dinman, Potomac, MD (US); Jeffrey J. DeStefano, Crofton, MD (US)

(21) Appl. No.: 12/757,182

(22) Filed: Apr. 9, 2010

Related U.S. Application Data
(63) Continuation of application No. PCT/US2008/079452, filed on Oct. 10, 2008.
(60) Provisional application No. 60/979,175, filed on Oct. 11, 2007.

(51) Int. Cl. A61K 38/12; A61K 31/7036; A61K 31/351; A61K 31/7008; A61K 31/7042; A61P 31/12; A61P 31/14

(52) U.S. Cl. 514/3.7; 514/40; 514/459; 514/61; 514/39; 514/37; 514/62

(57) ABSTRACT

The invention is directed to a method of stimulating PRF in a viral cell by administering an aminoglycoside antibiotic to said cell. In another embodiment, the invention is directed to a method of inhibiting viral replication by administering an aminoglycoside antibiotic to a viral cell. The invention is also directed to method of treating a viral infection in a patient suffering therefrom comprising administering to said patient an aminoglycoside antibiotic.

### RELATED APPLICATION

This application is a continuation of International Application No. PCT/US2008/079454, which designated the United States and was filed on Oct. 10, 2008, published in English, which is related to U.S. Provisional Application No. 60/979,175, filed on Oct. 11, 2007. The entire teachings of the above application are incorporated herein by reference.

### GOVERNMENT SUPPORT

The invention was supported, in whole or in part, by grant R01 GM058859 from the National Institute of General Medicine. The Government has certain rights in the invention.

### BACKGROUND OF THE INVENTION

Many viruses, such as HIV-1 and SARS-CoV, require ribosomes to shift reading frame at a specific frequency in order to produce the correct ratios of viral proteins required for virus particle assembly. One mechanism by which a reading frame can be shifted is referred to as programmed ribosomal frameshifting (PRF). PRF is utilized by RNA viruses, typically those with a positive (+) strand and double-stranded RNA (dsRNA) genomes, and by retroviruses and retroelements (Dinman et al., Chapter 22, Translational Control in Biology and Medicine, Cold Spring Harbor Press, Cold Spring Harbor, N.Y., 2006). By adjusting the reading frame of mRNA, PRF allows the usual stop codon to be bypassed by shifting the ribosome out of frame by a single nucleotide. As such, a single mRNA transcript can encode both a non-frameshift-encoded protein and a longer, frameshift-encoded fusion protein (Dinman et al., 1998). As a result, PRF enables viral cells to pack more information into their genomes than they could otherwise (Dinman et al., 1998). The frequency of frameshifting is a function of kinetic partitioning between the rates of the forward reaction (e.g., remaining in frame) and rates of the side reaction (e.g., shifting into the new frame).

A common PRF viral strategy entails inducing a net shift in reading by one base in the 5′ direction (referred to as −1 PRF). The best understood −1 PRF signal comprises three parts, a heptameric “slippery site,” a spacer and a strong secondary mRNA structure, typically an mRNA pseudoknot (Brierly (1995), J. Gen. Virol. 76: 1885-92). Prior to the frameshift, the interaction between tRNA and the mRNA codon in the ribosomal decoding center is termed “cognate,” meaning that bases in all three positions are paired with one another. Subsequent to a PRF event, only the first two bases in the codon:anticodon interaction are fully paired and the third is only weakly paired. This is termed a “near-cognate” tRNA:mRNA interaction. Such near-cognate interactions are recognized by the ribosome as subobtimal and are proofread at a significant rate (Daviter et al., 2006). A fraction of the shifted tRNAs are then rejected by the ribosome. Stabilizing near-cognate interactions is believed to inhibit rejection by the normal proofreading activity of the ribosome and thus, increase the frequency of productive PRF events which, in turn, alters the ratio of viral proteins and results in a collapse of viral particle assembly. PRF is thus considered a promising target for antiviral therapy (Dinman et al., 1992; Dinman et al., 1998) and it would be advantageous to identify agents that stimulate productive PRF events.

### SUMMARY OF THE INVENTION

The present invention is based on the discovery that aminoglycoside antibiotics decrease proofreading of shifted ribosomes resulting in a net increase in productive PRF events. For example, as shown in Example 1, administration of the aminoglycoside antibiotic, gentamicin, increased the PRF rate by about 40% in both HeLa and Jurkat cells.

In one embodiment of the invention, the invention is directed to method of treating a viral infection in a patient suffering therefrom comprising administering to said patient an aminoglycoside antibiotic. In another embodiment, the aminoglycoside antibiotic is added in an amount sufficient to increase the frequency of productive PRF events.

In another embodiment, the present invention is directed to a method of increasing the frequency of productive PRF events by a ribosome comprising administering an aminoglycoside antibiotic to a viral-infected cell. In another embodiment, the invention is directed to a method of inhibiting viral replication comprising administering an aminoglycoside antibiotic to a virus-infected cell. In another embodiment, the aminoglycoside antibiotic is administered in an amount sufficient to increase the frequency of productive PRF events.

In a further embodiment, the aminoglycoside antibiotic is one that is capable of interacting with a eukaryotic ribosome.

In yet another embodiment, the aminoglycoside antibiotic is gentamicin or a gentamicin derivative.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a drawing depicting cognate codon:anticodon interactions and near-cognate interactions.

FIG. 2A is a bar graph showing the percent increase in PRF for −1 PRF promoted by the endogenous yeast signal L-A virus signal and +1 PRF promoted by the Ty1 retrotransposable element signal in the presence (500 ug/ml) or absence of gentamicin.

FIG. 2B is a bar graph showing the increase in −1 PRF promoted by the HIV-1 frameshift signal in HeLa and Jurkat cells cultured in 5, 25, 50, 250 and 500 ug/ml gentamicin.

FIG. 3A is a bar graph showing the percent of Killer⁺ yeast cells passaged every 24 h in liquid medium containing 0, 50 or 500 ug/ml at 0, 2, 4, 6 and 8 days.

FIG. 3B is a picture of Killer assay colonies from drug treated (K⁻) and control cells (K⁺).

FIG. 3C is a picture of a TAE agarose gel stained with ethidium bromide in which RNA extracted from Killer⁺ control and Killer⁻ gentamicin treated yeast cells were separated.

FIG. 4A is a bar graph showing reverse transcriptase (RT) activity (colonies per million×10⁵/ml) in Jurkat E6-1 cells infected with HIV-1 in media containing gentamicin at 0, 50, 250 or 500 ug/ml at 2, 4, 6, 8 and 10 days post-infection.

FIG. 4B is a plot of the level of infectious virus (TCID₅₀/ml) in cells treated with 0, 50 and 250 ug/ml gentamicin for 10 days post-infection.

FIG. 5 is a bar graph showing the increase in −1 PRF promoted by the HIV-1 and SARS-Co-V frameshift signals in HeLa and Jurkat cells at 5, 25, 50, 250 and 500 ug/ml gentamicin.

### DETAILED DESCRIPTION OF THE INVENTION

The present invention is directed to methods of increasing the frequency of productive programmed ribosomal frameshifting (PRF) events by a ribosome comprising treatment of a virus-infected cell with an aminoglycoside antibiotic. The invention is also directed to methods for the treatment of a viral infection in a patient comprising administering to said patient an aminoglycoside antibiotic.

As used herein, the words “a” and “an” refer to one or more unless otherwise specified.

The terms “programmed ribosomal frameshifting” and “PRF” encompass frameshifting in either the 5′ or 3′ direction of the mRNA. In one embodiment, the aminoglycoside antibiotic stimulates −1 PRF. As used herein, “−1 PRF” refers to a net shift in reading by one base in the 5′ direction of the mRNA.

In certain embodiment of the inventive methods, the aminoglycoside antibiotic is administered to the virus-infected or to the patient in an amount sufficient to increase the frequency of productive programmed ribosomal (PRF) events. The phrases “to increase the frequency of productive programmed ribosomal frameshifting (PRF) events,” “increasing the frequency of productive programmed ribosomal frameshifting (PRF) events” and “increasing productive programmed ribosomal frameshifting (PRF) events” are intended to encompass increasing the incidence of shifted RNA species by inhibiting proofreading by the ribosome.

In certain other embodiments of the inventive methods, the aminoglycoside antibiotic is administered to the virus-infected cell or to the patient in amount sufficient to inhibit proofreading by the ribosome. In certain aspects, the ribosome is a eukaryotic ribosome.

Aminoglycoside antibiotics are a group of bactericidal antibiotics derived from the species of Streptomyces or Micromonosporum and are characterized by two or more amino sugars joined by a glycoside linkage to a central hexose. In bacteria, aminoglycosides act by causing misreading and inhibition of protein synthesis on bacterial ribosomes. The clinical utility of most aminoglycosides is based on differences in the nucleotide sequences in prokaryotic and eukaryotic ribosomes. Because aminoglycoside antibiotics are specific for prokaryotic ribosomes, treatment with this class of antibiotics results in misreading of bacterial RNA while not affecting eukaryotic protein synthesis. However, the ototoxicity and renal toxicity associated with some aminoglycoside antibiotics suggests that at least some aminoglycoside antibiotics have an effect on human ribosomes.

In one embodiment, the aminoglycoside antibiotic is an aminoglycoside antibiotic that does not contain paromamine. In another embodiment, the aminoglycoside antibiotic is an aminoglycoside antibiotic other than paromomycin or a pharmaceutically acceptable salt thereof. In yet another embodiment, the aminoglycoside antibiotic is selected from the group consisting of amastatin, amikacin, arbekacin, astromycin, bekanamycin, butirosin, daunorubicin, dibekacin, dihydrostreptomycin, fradiomycin, G 418, gentamicin, hygromycin, isepamicin, kanamycin, kirromycin, micronomicin, neomycin, netilmicin, ribostamycin, sisomycin, spectinomycin, streptomycin, streptozocin, thiostrepton, tobramycin, derivatives thereof and pharmaceutically acceptable salts thereof. Pharmaceutically acceptable salts of aminoglycoside antibiotics include inorganic salts (e.g., hydrochloride, sulfate, and the like) and organic salts (e.g., acetate). In a further embodiment, the aminoglycoside antibiotic is gentamicin or a derivative thereof. Neomycin includes, but is not limited to, neomycin A (also referred to as neamine), neomycin B and neomycin C. An exemplary neomycin derivative is 6′-N-acetyl neomycin B. Hybrimycin includes, but is not limited to, hybrimycin A1, hybrimycin A2, hybrimycin B1 and hybrimycin B2. Kanamycin includes, but is not limited to, kanamycin A, kanamycin B and kanamycin C. Exemplary kanamycin derivatives are 6′-N-acetyl kanamycin A, and 6′-N-acetyl kanamycin B. Gentamicin includes, but is not limited to, gentamicin A, gentamicin B gentamicin C₁ₐ, gentamycin C₁ and gentamicin C₂. Exemplary gentamycin derivatives are 2′-N-acetyl gentamicin C₁ₐ, 6′-N-acetyl gentamicin C₁ₐ, 3-N-acetyl gentamicin C₁ₐ, and gentamicin C₁ₐ adenylate.

In one embodiment, the aminoglycoside antibiotic used according to a method of the invention is one that is capable of interacting with a eukaryotic ribosome. Methods of identifying antibiotics that interact with eukaryotic ribosomes will be apparent to one of skill in the art. One such method includes determining whether the growth of eukaryotic cells is inhibited upon administration of a candidate aminoglycoside antibiotic. Another method of identifying aminoglycoside antibiotics that interact with eukaryotic ribosomes comprises labeling a candidate aminoglycoside antibiotic (for example, using a radioactive or fluorescent label), mixing the labeled candidate antibiotic with eukaryotic ribosomes followed by purifying the ribosomes. Co-purification of the candidate antibiotic with the purified ribsomes provides evidence of a physical interaction between the candidate antibiotic and the eukaryotic ribosome. A physical interaction between a candidate aminoglycoside antibiotic and a eukaryotic ribosome can also be detected using Biacore technology.

In one embodiment, the aminoglycoside antibiotic that is capable of interacting with a eukaryotic ribosome is selected from the group consisting of netilmicin, tobramycin, neomycin, gentamicin, hygromycin, G418 and pharmaceuteutically acceptable salts thereof.

In another embodiment, the invention is directed to methods of inhibiting viral replication comprising administering to a virus-infected cell an aminoglycoside antibiotic. In one embodiment, the virus is an RNA virus. The methods of the invention are useful for inhibiting viral replication in any virus that uses the PRF mechanism. The invention is also directed to a method of treating a viral infection in a patient in need thereof comprising administering to the patient an aminoglycoside antibiotic in an amount sufficient to increase the frequency of productive PRF events. In addition, the methods of the invention are useful for treating a viral infection wherein the virus uses −1PRF.

In yet another embodiment, the invention is a method of decreasing viral titer in a patient in need thereof comprising administering to the patient an aminoglycoside antibiotic.

In one embodiment, the virus is an RNA virus. In another embodiment, the RNA virus is a single-stranded (ss) virus with positive (+) sense strand or a double-stranded (ds) RNA virus. Single-stranded viruses with a (+) sense strand include, but are not limited to a virus from a family selected from the group consisting of Astroviridae, Coronaviridae, Caliciviridae, Flavivirdae, Picornaviridae and Togaviridae and a virus from the genus Hepevirus. Viruses from the family Coronoviridae include the human coronaviruses, such as the SARS-Associated Coronavirus, 229-E, OC43; animal coronaviruses, such as calf coronavirus, transmissible gastroenteritis virus of swine, hemagglutinating encephalomyelitis virus of swine, and porcine epidemic diarrhea virus; canine coronavirus; feline infectious peritonitis virus and feline enteric coronavirus; infectious bronchitis virus of fowl and turkey bluecomb virus; mouse hepatitis virus, rat coronavirus, and rabbit coronavirus. Similarly, torovirus (a type of coronavirus) is included, such as human toroviruses associated with enteric and respiratory diseases; breda virus of calves and bovine respiratory virus; berne virus of horses; porcine torovirus; feline torovirus. Another coronavirus is the arterivirus, which includes simian hemorrhagic fever virus, equine arteritis virus, Lelystad virus (swine), VR2332 virus (swine), and lactate dehydrogenase-elevating virus (rodents). An exemplary virus from the family Calciviridae is the Norwalk virus. Viruses from the Flaviviridae family include, for example, the Yellow Fever virus, West Nile virus, Hepatitis C virus and Dengue fever virus. Viruses from the Picornaviridae family include, for example, Polio Virus, the common cold virus and Hepatitis A Virus. A virus from the genus hepevirus includes, for example, Hepatitis E.

In yet another embodiment, the RNA virus is a dsRNA virus. Double-stranded RNA viruses include, for example, viruses from the Families Birnaviradae and Reoviridae (including, for example, rotavirus).

In a further embodiment the virus is a retrovirus. In one embodiment, the retrovirus is selected from the genus selected from the group consisting of alpharetrovirus, betaretrovirus, gammaretrovirus, deltaretrovirus, epsilonretrovirus, lentivirus and spumavirus. Lentiviruses (immunodeficiency viruses) include, for example, HIV-1 and HIV-2, SIV, FIV, BIV, Visna virus, Arthritis-encephalitis virus, and equine infectious anemia virus. Spumaviruses (the foamy viruses) include, for example, human foamy virus and other mammalian foamy viruses. Alphaviruses include, for example, avian leucosis viruses. Betaviruses, include, for example, mouse mammary tumor virus. Deltaviruses include, for example, T cell lymphotrophic viruses, such as HTLV-I, HTLV-II, STLVs, and BLV.

The methods of the invention are directed to administration of an aminoglycoside antibiotic in an amount sufficient to increase the frequency of productive PRF events in a viral cell. In one embodiment, the frequency of PRF events is increased by at least about 5%. As used herein, the percentage increase in PRF events is the percent increase in PRF events in virus-infected cells treated with an aminoglycoside antibiotic compared to the frequency of PRF events in cells not treated with the aminoglycoside antibiotic. In another embodiment, the frequency of PRF events is increased by at least about 10%. In another embodiment, the frequency of PRF events is increased by at least about 20%. In yet another embodiment, the frequency of PRF events is increased by at least about 30%. In a further embodiment, the frequency of PRF events is increased by at least about 40%.

The methods of the present invention are particularly suited to treatment of any animal, particularly a mammal, and more specifically human. As used herein, the term “patient” encompasses any animal. Animals to be treated include but are not limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., i.e., for veterinary medical use. In one embodiment, the patient is a human.

In one embodiment, the amount of aminoglycoside antibiotic that is to be administered to a patient suffering from a viral infection is an amount sufficient to increase the frequency of productive PRF events. In another embodiment, the amount of aminoglycoside antibiotic that is to be administered to a patient suffering from a viral infection is an amount sufficient to decrease proofreading by the ribosome. In yet another aspect of the invention, the amount of aminoglycoside antibiotic that is to be administered to a patient suffering from a viral infection is a therapeutically effective amount. The term “therapeutically effective amount” as used herein means an amount of aminoglycoside antibiotic that is effective, at dosages and for periods of time necessary, to prevent, diminish, inhibit or eradicate symptoms of viral infection, in a patient. As used herein, the term “therapeutically effective amount” also encompasses an amount of aminoglycoside antibiotic sufficient to increase the frequency of productive programmed ribosomal frameshifting events and/or an amount of aminoglycoside antibiotic sufficient to decrease proofreading by the ribosome. A therapeutically effective amount of a composition of the invention may vary according to factors such as the disease state, age, sex and weight of the individual. Dosage regimes may be adjusted to provide the optimum therapeutic response. For example, several divided doses may be administered daily or the dose may be proportionally reduced as indicated by the exigencies of the therapeutic situation. Methods of administering an aminoglycoside antibiotic will be appreciated by one of skill in the art. Methods of administration include, for example, parenteral, transmucosal, transdermal, intramuscular, intravenous, intradermal, subcutaneous, intraperitoneal, intraventricular, intracranial, oral administration or administration by inhalation.

As is appreciated by those skilled in the art, the amount of the compound may vary depending on its specific activity and suitable dosage amounts may range from about 0.1 to 20, preferably about 0.5 to about 10, and more preferably one to several milligrams of active ingredient per kilogram body weight of individual per day and depend on the route of administration. In one embodiment the amount is in the range of 10 picograms per kg to 20 milligrams per kg. In another embodiment the amount is 10 picograms per kg to 2 milligrams per kg. In another embodiment the amount is 2-80 micrograms per kilogram. In another embodiment the amount is 5-20 micrograms per kg. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

As described below in Example 1, increased −1 PRF was observed at drug concentrations that are normally used in cell culture. This indicates that the dose of aminoglycoside antibiotic necessary to increase frameshifting is less than that required to inhibit cell growth.

The aminoglycoside antibiotic can be administered in a pharmaceutical composition comprising the aminoglycoside antibiotic and a pharmaceutically acceptable carrier. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and other animals orally, rectally, parenterally, intracisternally, intravaginally, intraperitoneally, topically (as by powders, ointments, or drops), bucally, or as an oral or nasal spray.

Liquid dosage forms for oral administration include pharmaceutically acceptable emulsions, microemulsions, solutions, suspensions, syrups and elixirs. In addition to the aminoglycoside antibiotics, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, powders, capsules, pills, and granules can be prepared with coatings and shells such as enteric coatings and other coatings well known in the pharmaceutical formulating art. They may optionally contain opacifying agents and can also be of a composition whereby they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### EXAMPLES

### Example 1

### A New Application of Aminoglycoside Antibiotics for Treatment of Viral Diseases

### Abstract

Many RNA viruses use programmed −1 ribosomal frameshifting (−1 PRF) as a genome condensation strategy to synthesize large quantities of structural proteins and smaller quantities of enzymatic products. The frequency of −1 PRF determines the ratio of structural/enzymatic proteins. Changes in −1 PRF efficiency alter these ratios and significantly interfere with viral particle assembly suggesting −1 PRF as a target for antiviral therapeutics. The frameshift event results in a near-cognate codon:anticodon interaction in the ribosomal A-site. This class of tRNA:mRNA interactions are substrates for translational proofreading. Small molecules capable of stabilizing such complexes should increase the frequency of productive −1 PRF events, and thus have antiviral effects. Here we demonstrate that gentamicin, a clinically proven, inexpensive and orally available aminoglycoside antibiotic, stimulates −1 PRF as directed by two different viral signals and that it has antiviral activity against both a endogenous dsRNA virus of yeast, and on HIV-1 in cell culture. The potential implications of these findings on the HIV/AIDS epidemic are discussed.

### Introduction

Between 33.4 million and 46 million people worldwide have HIV, with close to 25 million in sub-Saharan Africa alone, and in 2005, the estimated total worldwide expenditures on HIV/AIDS treatment and awareness was approximately $8 billion¹. Many aspects of the virus and the disease have made controlling this epidemic extremely difficult. Despite its ability to provoke a strong immune response, the virus effectively evades immune clearance, severely hampering vaccine development efforts. Development of new antiviral drugs takes a long time, resulting in high treatment costs, effectively preventing their widespread use by the impoverished people who need them most. For those who can afford them, these drugs can also have severe side effects, raising significant quality of life issues. The currently available antiretroviral drugs also have limited efficacy, as they provide agents of selective pressure for evolution of drug resistant quasispecies. A cheap accessible treatment that circumvents the normal evolution of drug resistance could completely change the course of the epidemic.

The very small volumes of viral particles enforce stringent limits on genome size. A consequence of this is that viral genomes have evolved numerous condensation strategies. A response to this has been the evolution of overlapping reading frames (ORFs) and of cis-acting mRNA elements that direct a fraction of elongating ribosomes to shift from the upstream into the downstream ORF. One such strategy, called “Programmed Ribosomal Frameshifting” (PRF) is often used by RNA viruses, typically those with (+) strand and dsRNA genomes, as well as by retroviruses and retroelements (reviewed in Ref. 2). Importantly, the frequency the PRF event determines the stoichiometric ratios between the two gene products. Measurements of PRF efficiencies have shown that these have evolved to produce the optimum ratios of viral proteins required for viral particle morphogenesis, and that alterations of these rates have strong negative impacts on virus propagation, thus identifying PRF as a potential target for antiviral therapeutics (reviewed in Ref. 3).

One common PRF viral strategy involves inducing a net shift of reading by one base in the 5′ or −1 direction, called −1 PRF. The best understood −1 PRF signals are tripartite, consisting of a hepatmeric “slippery site”, followed by a “spacer” which in turn is followed by a strong secondary mRNA structure, typically an mRNA pseudoknot (reviewed in Ref. 4). The slippery site has been defined as the sequence X XXY YYZ, in which XXX is any three identical nucleotides, YYY can be either UUU or AAA, and Z≠G. Although some controversy remains as to the specific mechanistic details of −1 PRF, the general framework of the original “simultaneous slippage” model⁵ still is widely accepted. By this model, the role of the downstream secondary structure is to cause elongating ribosomes to pause with their A- and P-site tRNAs positioned over bases 2-7 of the slippery site. The nature of the tRNA:mRNA interactions is such that, upon slippage by one base in the 5′ direction, new codon:anticodon interactions can be established by base pairing between the non-wobble bases of the tRNAs and the −1 frame codons. Evidence exists suggesting that −1 PRF can occur during/after aa-tRNA accommodation into the A-site but before peptidyltransfer (reviewed in Refs. 6, 7) and/or during translocation^{8,9}. The two models are not necessarily exclusive. Assuming the former model, comparison of the 0- and −1 frame codons in the functional slippery sites with the allowed 0-frame aa-tRNA anticodons reveals that the mRNA:tRNA interaction in the A-site after the shift should not be a cognate codon:anticodon couple. Rather, the requirement that the seventh base (Z) cannot be a G forces the A-site wobble base to be either a G, U, or A, any of which can participate in either standard or non-Watson-Crick H-bonding interactions with either the A or U base at the third position of the −1 frame A-site codon. The possibility for such interactions has recently been shown to be the functional requirement for near-cognate codon:anticodon interactions¹⁰. Near-cognate codon:anticodon interactions are substrates for translational proofreading (reviewed in Ref. 11). Thus, a fraction of shifted tRNAs should be rejected by the ribosome. Indeed, the original protein sequence data used to define the first frameshift signal hinted at this: although 70% of the transframe protein produced by the HIV-1 U UUU UUA slippery site was encoded by both the P- and A-site 0-frame codons (i.e. Phe Leu), 30% was encoded by the 0-frame P-site codon (Phe) and the −1 frame A-site codon (Phe)¹².

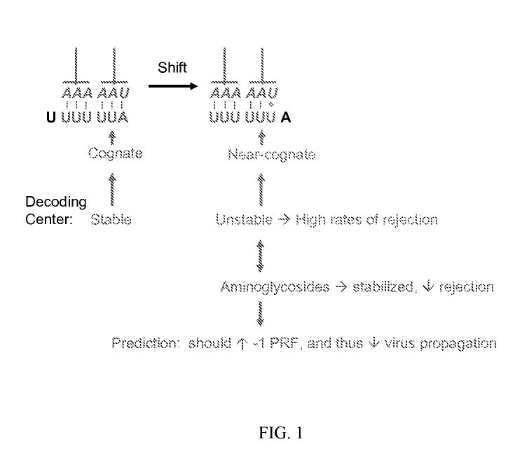

Aminoglycoside antibiotics function by binding to and displacing bases of the small subunit rRNA located in the ribosomal decoding center, forcing them to help stabilize near-cognate codon:anticodon mini-helices¹³. Thus, it is theoretically possible that aminoglycosides could be used to inhibit proofreading of shifted ribosomes, thus stimulating −1 PRF. The resulting increased production of frameshifted protein products would alter the ratios between viral proteins, with negative consequences on viral particle morphogenesis. This model is diagrammed in FIG. 1.

A practical problem with this approach is that the clinical utility of most aminoglycoside antibiotics is based on the differences in the nucleotide sequences between prokaryotes and eukaryotes, and their specificity for prokaryotic ribosomes forces misreading and accumulation of errors in bacteria while not affecting eukaryotic protein synthesis¹⁴. However, some aminoglysocides can interact with eukaryotic ribosomes and affect their fidelity. The uncommon, but well documented oto- and renal-toxicity of gentamicin suggests that this drug may affect human ribosomes. This is supported by biochemical evidence that selected aminoglycosides including gentamicin increased rates of translational misreading by mammalian and ciliate ribosomes in vitro^{15,16}. Further, deletion of the yeast genes encoding components of the ribosome-associated molecular chaperone complexes (RAC) SSZ1 and ZUO1 as well HSP70-like chaperones SSB1 and SSB2 results in altered rates of −1 PRF, but not +1 PRF¹⁷, and some of these same mutants have also been shown to be hypersensitive to gentamicin^{18,9}. Thus, gentamicin provides a likely starting point to test the hypothesis that some aminoglycosides should have stimulatory effects on −1 PRF, and thus have antiviral properties.

### A. Gentamicin Stimulates −1 PRF

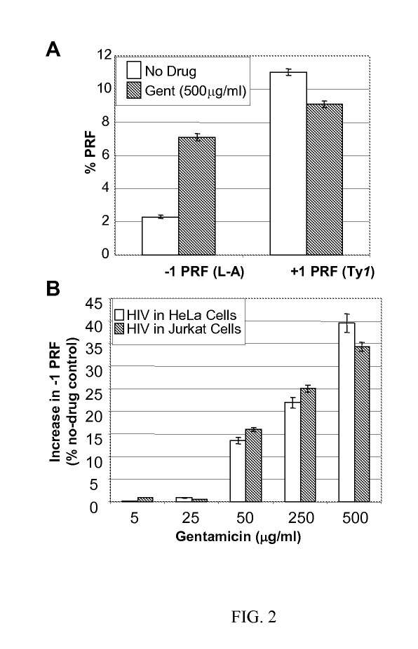

Frameshifting in yeast was monitored by using the dual luciferase reporter plasmid pYDL-LA (which contains an L-A virus derived −1PRF signal) and pYDL-Ty1 (which contains a Ty1 derived +1PRF signal)²⁰ in the presence or absence of gentamicin (500 μg/ml). The presence of the drug did not noticeably affect cell growth. As shown in FIG. 2A, gentamicin caused a >3-fold increase in L-A, virus promoted −1 PRF but had virtually no effect on Ty1 directed +1 PRF. Thus, similar to the loss of the RAC complex or Ssb function, the presence of gentamicin specifically affected −1 PRF in yeast. To test the generality of this hypothesis, the frameshifting studies were extended to assays in human-derived cells using reporter plasmids harboring the HIV-1 −1 PRF signal²¹. These studies employed HeLa CD4⁺ I and Jurkat cells transiently transfected with pJD175c (harboring the HIV −1 PRF signal) or pJD175d (0-frame control)²¹. Gentamicin stimulated HIV-1 directed −1 PRF by up to 40% in both cell lines (FIG. 2B). Importantly, increased −1 PRF was observed at drug concentrations that are normally used in cell culture (50 μg/ml).

### B. Gentamicin Inhibits Replication of the Yeast Killer Virus and of HIV-1

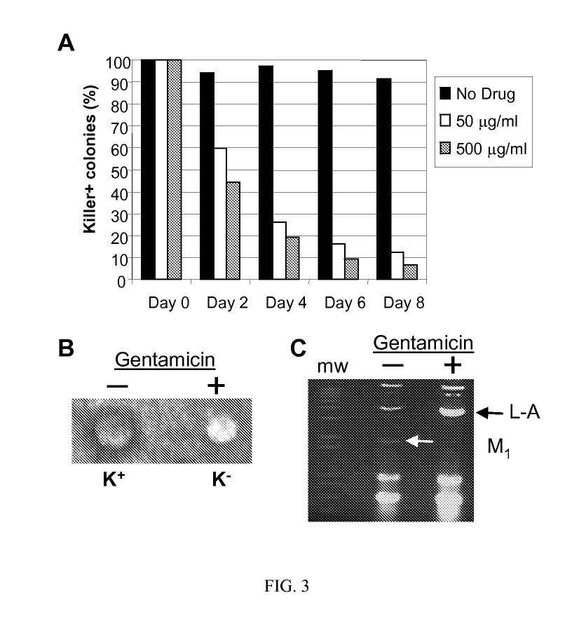

To test the hypothesis that gentamicin should have antiviral activity by stimulating −1 PRF, the effects of gentamicin were assayed on propagation of the endogenous yeast “Killer” virus and on HIV-1 replication in human cell culture. The yeast based experiments examined the effects of the drug on the ability of cells to propagate the endogenous “Killer” virus as previously described²² after serial passage in media containing two different drug concentrations (50 μg/ml and 500 μg/ml). As shown in FIG. 3A, gentamicin promoted rapid loss of the Killer phenotype at both drug concentrations. A representative yeast killer assay is shown in FIG. 3B. The Killer phenotype is due to the presence of two endogenous dsRNA viruses: the L-A helper virus that uses the −1 PRF to produce its Gag-pol fusion protein, and the M₁ satellite virus with encodes the secreted toxin responsible for the actual phenotype (reviewed in Ref, 23). M₁ is much more sensitive to changes in −1 PRF (and in ribosome function in general) than is L-A^{24,25}. To determine whether gentamicin affected maintenance of either L-A and/or M₁, dsRNA was preferentially extracted from Killer⁺ control, and gentamicin treated Killer⁻ cells and separated through a 1.2% TAE-agarose native gel as previously described²¹. The results show that the M₁ dsRNA genome was no longer present in the gentamicin treated cells (FIG. 3C) demonstrating that the gentamicin induced loss of the Killer phenotype was due to loss of the M₁ killer virus. Note that approximately 10-times more RNA was examined in the drug treated sample as compared to the no-drug control in an effort to detect lower amounts of the M₁ dsRNA.

### C. Effect of Gentamicin of HIV-1 Replication

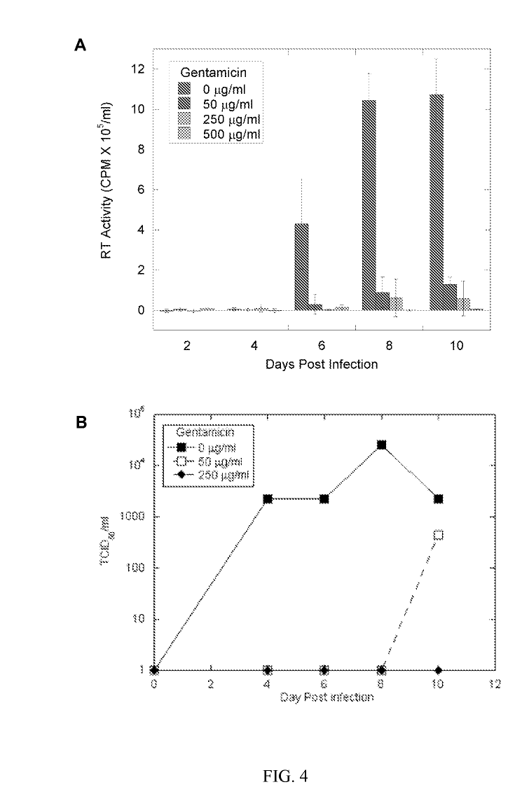

The effect of gentamicin on HIV-1 replication was also tested. Jurkat E6-1 cells were infected with HIV-1 strain LAI in the presence and absence of increasing concentrations of Gentamicin, and were assayed for the presence of virus every second day for a total of ten days. The results of a representative experiment are shown in FIG. 4A. In untreated cells, reverse transcriptase activity (RT) was clearly detectable by day 6, increased on day 8, and remained stable through day 10. In contrast, RT activity was only slightly detectable after 6 days in cells cultured at 50 μg/ml Gentamicin. Notably, this is the drug concentration typically used for cell culture, and this dose had no noticeable effects on cell growth or morphology. RT activities were even lower at higher drug concentrations, although significant cell toxicity was observed at 500 μg/ml. Control experiments showed that gentamicin did not affect HIV-1 RT activity. To independently monitor antiviral activity, the presence of HIV-1 viral particles in cell-free supernatants was directly titered using an HIV-1 p24 Antigen Capture Assay Kit (FIG. 4b). In the absence drug new viruses were detectable by day 4 and the titer rose through day 8, decreasing some at day 10. In contrast no infectious virus was made when 250 μg/ml of gentamicin was included in the media while a small amount of virus was detectable at day 10 only when 50 μg/ml was used. This was consistent with a small increase in the RT activity at this concentration of drug on day 10 (FIG. 4a).

### Concluding Remarks

The current work is based on the fact that frameshifting results in the presence of an near-cognate aa-tRNA:mRNA interaction in the ribosomal decoding center, and that aminoglycosides stabilize these complexes, inhibiting their rejection by the normal proofreading activity of the ribosome. Thus, aminoglycosides that are capable of interacting with eukaryotic ribosomes should increase the fraction of productive frameshift events, resulting in a net increase in the ratio of viral enzymatic to structural proteins produced. This in turn should interfere with viral particle morphogenesis programs, resulting in decreased rates of virus production. Gentamicin was chosen as a lead aminoglycoside compound based on its know effects on eukaryotic ribosomes, and we have shown that it stimulates −1 PRF directed by two different viral frameshift signals, and that it inhibits propagation of an endogenous yeast dsRNA virus as well as HIV-1 in human-derived cell culture.

FIG. 1 is a diagram depicting the theoretical basis for the link between increased −1PRF and decreased virus propagation. Top left: tRNAs are positioned at the “slippery site” of the −1 PRF signal in the incoming (0) frame. The cognate codon:anticodon interaction in the ribosomal decoding center is stable (indicated by blue). After the shift, the tRNAs are base paired to the −1 frame slippery site codons. The near-cognate codon:anticodon interaction in the decoding center is unstable, and is a substrate for translational proofreading (indicated in red). Aminoglycoside antibiotics stabilize this type of interaction, decreasing proofreading rates (purple). This engenders the hypothesis that aminoglycosides should increase the frequency of productive −1 PRF events, thus interfering with viral particle assembly and virus propagation.

FIG. 2 are bar graphs showing the effects of gentamicin on PRF. (A) −1 PRF promoted by the endogenous yeast L-A viral signal and +1 PRF promoted by the Ty1 retrotransposable element signal were monitored in yeast cells in the presence (500 μg/ml) or absence of gentamicin. (B) −1 PRF promoted by the HIV-1 frameshift signal was monitored in both HeLa and Jurkat cells cultured in the indicated concentrations of gentamicin.

FIG. 3 shows that gentamicin promotes loss of the endogenous yeast “Killer” virus. (A) Killer⁺ yeast cells harboring the L-A helper and M₁ satellite viruses were serially passaged every 24 hours in liquid medium containing 50 μg/ml, 500 μg/ml, or no drug for a total of eight days. At two day intervals, samples of cells were from each group were harvested, and streaked onto solid medium for single colonies. After 3 days of growth at 30° C., these were then replica plated onto 4.7 MB killer indicator plates that had been freshly seeded with a lawn of 5×47 killer indicator cells, and incubated at 20° C. for 5 days. The presence of the Killer phenotype was determined by the appearance of a lawn of growth inhibition around infected colonies. Killer maintenance was determined by dividing the number of Killer⁺ colonies by the total number of colonies. (B) Example of Killer assay using colonies from drug treated (K⁻) and control (K⁺) cells. The killer phenotype is indicated by a zone of growth inhibition around cells harboring the virus. (C) Gentamicin cures cells of the M₁ satellite virus. Total RNA was extracted from Killer⁺ control, and Killer⁻ gentamicin treated yeast cells, separated through a native 1.2% TAE agarose gel and stained with ethidium bromide. The L-A and M₁ dsRNA genomes are indicated. Note that the gentamicin treated Killer sample contained ˜10-fold more RNA than the Killer+ control in order to demonstrate that the M₁ dsRNA was not present.

FIG. 4 shows that gentamicin inhibits HIV-1 production in Jurkat cells. (A) Jurkat E6-1 cells were grown in RPMI-1640 media supplemented with 10% FBS. Cells were infected with HIV-1 (LAI) at an MOI of 0.01 in media containing gentamicin at 0, 50, 250, or 500 μg/ml. Infected or mock infected cells were maintained in the above media containing the corresponding concentration of gentamicin. Clarified viral supernatant was assayed for RT activity on days 2, 4, 6, 8, and 10 post infection. Significant cell toxicity was observed only at 500 μg/ml gentamicin. Results are from an average of 3 experiments with error bars corresponding to standard deviation values. (B) Clarified viral supernatants from days 4, 6, 8, and 10 above were used to perform limit-dilution assays in order to determine the level of infectious virus produced in untreated and gentamicin treated cells. A p24 antigen capture assay was used to detect virus. Results are reported as TCID₅₀/ml vs. day post infection. The antigen capture assay was repeated with similar results while the RT activity assay was repeated twice with similar results.

### Materials and Methods

Cells and viruses. Jurkat E6-1 cells (obtained through the ATCC) were cultured in RPMI-1640 supplemented with 10% fetal bovine serum at 37° C. and 5% CO₂ according to the ATCC data sheet. HIV-1 strain LAI was obtained through the NIH AIDS Research and Reference Reagent Program. Expanded virus stocks were titered on Jurkat cells using limit-dilution assays and an HIV-1 p24 Antigen Capture Assay Kit from AIDS Vaccine Program at NCI Frederick.

Frameshifting Assays. Yeast BY4741 (MATa his3Δ1 leu2Δ0 met15Δ0 ura3Δ0) transformed with pYDL-LA, pYDL-Ty1, or pYDL-control²⁰ were grown overnight in the presence or absence of gentamicin (500 μg/ml). Cell extracts were prepared and frameshifting was measured as previously described^{20,26}. To monitor frameshifting in human cells, HeLa CD4⁺ cells were plated at a concentration of 3.0×10⁵cells/ml and Jurkat cells at a concentration of 4.0×10⁵cells/ml in 24-well plates. The cells were cultured at 37° C. with 5% CO₂ in DMEM supplemented with 10% FBS and 0, 5 μg/ml, 25 μg/ml, 50 μg/ml, 250 μg/ml, or 500 μg/ml of gentamicin sulfate (Sigma Aldrich). The following day the cells were transiently transfected with 1 μg of pJD175c (harboring the HIV −1 PRF signal) or pJD175d (0-frame control)²¹ using 2 μl ExpressFect (Denville Scientific) per well following manufacturer's directions. Transfections were performed in triplicate per amount of gentamicin. After 24 hours, HeLa CD4⁺ cells were washed with PBS and lysed with 1× Passive Lysis Buffer (Dual-Luciferase Reporter System, Promega) with gentle rocking for fifteen minutes. Jurkat cells were harvested by centrifugation at 1000× for five minutes. The pellet was washed with PBS, and then cells were lysed by resuspension in 1× Passive Lysis Buffer. A Turner 20/20 luminometer was used to measure renilla and firefly luciferase activity (Dual-Luciferase Reporter System, Promega, Fitchburg, Wis., United States). The Renilla and firefly luciferase activities produced by the 0-frame control and −1 PRF test construct were measured for each of the gentamicin concentrations. A minimum of three independent luciferase readings were taken from each sample. Data were collected until normal distributions were established, allowing comparison across and between experiments as previously described²⁶.

Assays of antiviral activity of gentamicin in yeast. In yeast based experiments, Killer⁺ cells (JD932D: MATa ade 2-1 trp1-1 ura3-1 leu2-3,112 his3-11,15 can1-100 [L-AHN M₁]) were serially passaged every 24 hours in liquid medium containing 50 μg/ml, 500 μg/ml, or no drug for a total of eight days. At two day intervals, samples of cells were from each group were harvested, and streaked onto solid medium for single colonies. After 3 days of growth at 30° C., these were then replica plated onto 4.7 MB killer indicator plates that had been freshly seeded with a lawn of 5×47 killer indicator cells, and incubated at 20° C. for 5 days. The presence of the Killer phenotype was determined by the appearance of a lawn of growth inhibition around infected colonies. Killer maintenance was determined by dividing the number of Killer⁺ colonies by the total number of colonies. Total RNA was extracted from yeast cells, separated through a native 1.2% TAE agarose gel and visualized with ethidium bromide as previously described²².

HIV infection of Jurkat cells. All experiments were performed with Jurkat cells in 24-well plates and in duplicate. 2×10⁵ cells were infected at an MOI of 0.01 or mock infected by incubating cells and virus in 150 μl of complete medium containing 0, 50, 250, or 500 μg/ml of gentamicin (Sigma) for 1 h at 37° C. in 5% CO₂. Cells were subsequently washed once with 500 μl of RPMI-1640 then resuspended in complete medium (4×10⁵ cells/ml) containing gentamicin at the same concentration. Fifty total μl were removed for various assays (cell viability using trypan blue, virus titer determination by p24 antigen capture assay, and RT activity analysis) at 2, 4, 6, 8, and 10 days post infection. Cells were routinely supplemented with complete medium containing gentamicin. Mock infections with the same genatmicin concentrations were performed as controls.

Reverse transcriptase assay of supernatants from infected cells. Assays contained 10 μl of cell-free (clarified by centrifugation at 5000 rpm for 3 min in a microfuge) virus supernatant from infected Jurkats at the indicated time points. Twenty μl of assay buffer [50 mM Tris-HCl (pH=8), 75 mM KCl, 2 mM DTT, 5 mM MgCl₂, 0.05% NP-40, 2.5 μg poly(rA)-oligo(dT₂₀) (8:1 w:w rA:dT), and 5.6 μM ³H-dTTP (5.6 Ci/mmol)] was added to viral supernatant and incubated at 37° C. for 2 hours. The entire reaction was spotted onto a 25 mm DE-81 filter disk, dried, washed 3 times with 0.5 M Na₂PO₄, and a final time with 80% ethanol. Dried filters were counted using a scintillation counter. Control reactions including 20, 50, or 100 fmoles of HIV-RT (Worthington Biochemical) were also performed.

There are many advantages of aminoglycosides over current retrovirals. They are clinically proven, having been used to control bacterial infections in humans for over 60 years. They are generally well tolerated in humans, and their side effects are well known and generally treatable. Aminoglycosides are inexpensive to produce, most are off patent, stable and orally active. Importantly, gentamicin represents only a single lead compound; there are many more clinically approved aminoglycosides, and potentially millions more chemical variants in pharmaceutical company libraries. In addition, while other antivirals target viral proteins, aminoglycosides target essential host encoded molecular machinery. Typically, drug resistance evolves to reduce or eliminate ability of viral gene products to interact with the drug. In this case, the direct target of the drug to a host factor circumvents this strategy, and it would be difficult for viruses to evolve mechanisms to alter the interaction between the ribosome and drug. Instead, virus resistance will most likely evolve through mutations of −1 PRF signal that compensate for effects of drug on frameshifting efficiency, e.g. mutations that decrease intrinsic rates of −1 PRF. The consequence of this strategy is that resistant mutants should be dependent on the presence of a specific drug. Thus, virus populations that are resistant to one drug should be vulnerable to withdrawal of that drug, or substitution with another drug that stimulates or inhibits −1 PRF to a different degree.

### Example 2

### Effect of Gentamicin on HIV and SARS-CoV Mediated Programmed Ribosomal Frameshifting

The SARS-CoV frameshift signal was cloned into dual luciferase vectors as previously described in Plant et al., (PLoS Biol. 3: 1012-1023 (2005)), as both a 0-frame control (pJD464) and a −1 test construct (pJD502). The HIV −1 PRF signal (pJD175c) and 0-frame control (pJD175d) used for dual luciferase assays in mammalian cells were previously reported by Grentzmann et al., (RNA 4: 479-486 (1998)).

HeLa CD4+ cells were plated at a concentration of 3.0×10⁵cells/mL and Jurkat cells were plated at a concentration of 4.0×10⁵ cells/mL in a 24-well plate. The cells were cultured in DMEM supplemented with 10% FBS and increasing amounts of gentamicin sulfate (Sigma Aldrich) at 37° C. with 5% CO₂. The following day, the cells were transiently transfected with 1 μg of plasmid DNA using ExpressFect (Denville Scientific, Inc., South Plainfield, N.J.) according to the manufacturer's directions. After 24 hours, cells were lysed with Passive Lysis Buffer and a Turner 20/20 luminometer was used to measure renilla and firefly luciferase activity (Dual-Luciferase Reporter System, Promega, Fitchburg, Wis.). Transfections were performed in triplicates per amount of gentamicin used with at least three luciferase readings from each transfection. Data was collected until a normal distribution was established, allowing comparison across and between experiments.

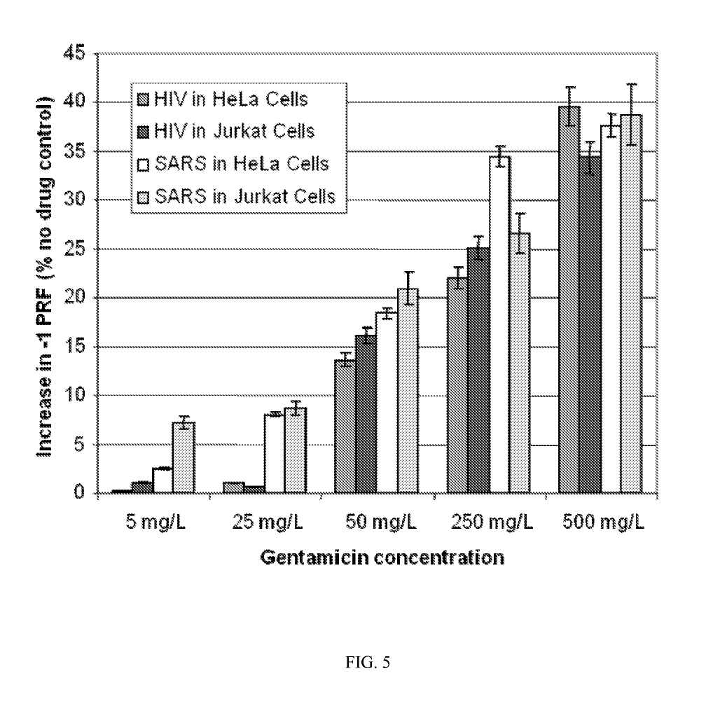

As shown in FIG. 5, gentamicin dose-dependently increased HIV-1 and SARS-CoV mediated −1 PRF in HeLa and Jurkat cells.

### REFERENCES AND NOTES

1. UNAIDS: United Nations 2006 Report on the global AIDS epidemic (UNAIDS, Geneva, Switzerland, ed. 10, 2007).

2. J. D. Dinman and M. J. Berry, in Translational Control in Biology and Medicine., M. B. Mathews, N. Sonenberg, J. W. B. Hershey, Eds. (Cold Spring Harbor Press, Cold Spring Harbor, N.Y., 2006), chap. 22.

3. J. D. Dinman, M. J. Ruiz-Echevarria, S. W. Peitz, Trends in Biotech. 16, 190-196 (1998).

4. I. Brierley, J. Gen. Virol. 76, 1885-1892 (1995).

5. T. Jacks, H. D. Madhani, F. R. Masiraz, H. E. Varmus, Cell 55, 447-458 (1988).

6. J. W. Harger, A. Meskauskas, J. D. Dinman, TIBS 27, 448-454 (2002).

7. E. P. Plant et al., RNA 9, 168-174 (2003).

8. P. A. Ortiz, R. Ulloque, G. K. Kihara, H. Zheng, T. G. Kinzy, J. Biol. Chem. 281, 32639-32648 (2006).

9. M. Leger, D. Dulude, S. V. Steinberg, L. Brakier-Gingras, Nucleic Acids Res. 35, 5581-5592 (2007).

10. E. P. Plant et al., PLoS ONE 2, e517 (2007).

11. T. Daviter, K. B. Gromadski, M. V. Rodnina, Biochimie 88, 1001-1011 (2006).

12. T. Jacks et al., Nature 331, 280-283 (1988).

13. A. P. Carter et al., Nature 407, 340-348 (2000).

14. M. I. Recht, S. Douthwaite, J. D. Puglisi, EMBO J. 18, 3133-3138 (1999).

15. M. O. Loveless, S. J. Kohlhepp, D. N. Gilbert, J. Lab Clin. Med. 103, 294-303 (1984).

16. D. C. Eustice and J. M. Wilhelm, Biochemistry 23, 1462-1467 (1984).

17. K. L. Muldoon-Jacobs and J. D. Dinman, Nucleic Acids Res. (2006).

18. M. C. Wagner, E. E. Molnar, B. A. Molitoris, M. G. Goebl, Antimicrob. Agents Chemother. 50, 587-595 (2006).

19. A. S. Blackburn and S. V. Avery, Antimicrob. Agents Chemother. 47, 676-681 (2003).

20. J. W. Harger and J. D. Dinman, RNA 9, 1019-1024 (2003).

21. J. D. Dinman et al., Proc. Natl. Acad. Sci. USA 99, 5331-5336 (2002).

22. J. D. Dinman, M. J. Ruiz-Echevarria, K. Czaplinski, S. W. Peitz, Proc. Natl. Acad. Sci. USA 94, 6606-6611 (1997).

23. R. B. Wickner, in The Molecular and Cellular Biology of the Yeast Saccharomyces: genome dynamics, proteins synthesis, and energetics., J. R. Broach, E. W. Jones, J. R. Pringle, Eds. (Cold Spring Harbor Press, Cdld Spring Harbor, N.Y., 1991), vol. 1, chap. 5.

24. J. D. Dinman and R. B. Wickner, Genetics 136, 75-86 (1994).

25. Y. Ohtake and R. B. Wickrier, Mol. Cell. Biol. 15, 2772-2781 (1995).

26. J. L. Jacobs and J. D. Dinman, Nucleic Acids Res. 32, e160-e170 (2004).

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating a viral infection in a patient suffering therefrom comprising treating said patient with an aminoglycoside antibiotic, wherein the aminoglycoside antibiotic is administered in an amount sufficient to increase the frequency of productive programmed ribosomal frameshifting events and wherein the aminoglycoside antibiotic is other than paromomycin.

2. The method of claim 1, wherein the aminoglycoside antibiotic interacts with a eukaryotic ribosome.

3. The method of claim 1, wherein said aminoglycoside antibiotic is selected from the group consisting of amastatin, amikacin, arbekacin, astromycin, bekanamycin, butirosin, daunorubicin, dibekacin, dihydrostreptomycin, fradiomycin, G 418, gentamicin, hygromycin, isepamicin, kanamycin, kirromycin, micronomicin, neomycin, netilmicin, ribostamycin, sisomycin, spectinomycin, streptomycin, streptozocin, thiostrepton and tobramycin and pharmaceutically acceptable salts thereof.

4. The method of claim 3, wherein the aminoglycoside antibiotic is selected from the group consisting of netilmicin, tobramycin, neomycin, gentamicin, hygromycin, G418 and pharmaceuteutically acceptable salts thereof.

5. The method of claim 4, wherein the aminoglycoside antibiotic is gentamicin or a pharmaceutically acceptable salt thereof.

6. The method of claim 1, wherein the aminoglycoside antibiotic is a gentamicin derivative.

7. The method of claim 1, wherein said viral infection is an RNA viral infection.

8. The method of claim 6, wherein the RNA virus is a single-stranded, positive sense RNA virus.

9. The method of claim 7, wherein the RNA virus is from a family selected from the group consisting of Coronaviridae, Calciviridae, Flaviviridae and Picornaviridae.

10. The method of claim 8, wherein the RNA virus is from the family Coronaviridae.

11. The method of claim 9, wherein the RNA virus is the SARS-coronavirus.

12. The method of claim 8, wherein the RNA virus is from the family Calciviridae.

13. The method of claim 11, wherein the RNA virus is the Norwalk virus.

14. The method of claim 8, wherein the RNA virus is from the family Flaviviridae.

15. The method of claim 13, wherein the RNA virus is selected from the group consisting of Yellow fever virus, West Nile virus, Hepatitis C virus and Dengue fever virus.

16. The method of claim 8, wherein the RNA virus is from the family Picornaviridae.

17. The method of claim 15, wherein the RNA virus is selected from the group consisting of the polio virus, common cold virus and hepatitis A virus.

18. The method of claim 8, wherein the RNA virus is from the genus of Herpe virus.

19. The method of claim 17, wherein the RNA virus is hepatitis E virus.

20. The method of claim 6, wherein the RNA virus is a double-stranded RNA virus.

21. The method of claim 19, wherein the RNA virus is rotavirus.

22. The method of claim 1, wherein the viral infection is a retroviral infection.

23. The method of claim 21, wherein the retrovirus is a lentivirus.

24. The method of claim 23, wherein the lentivirus is selected from the group consisting of HIV-1 and HIV-2.

25. The method of claim 23, wherein the lentivirus is HIV-1.

26. The method of claim 24, wherein the aminoglycoside antibiotic is gentamicin or a pharmaceutically acceptable salt thereof.

27. The method of claim 1, wherein the frequency of productive PRF events is increased by at least about 5%.

28. The method of claim 27, wherein the frequency of productive PRF events is increased by at least about 20%.

29. The method of claim 28, wherein the frequency of productive PRF events is increased by at least about 40%.

30. The method of claim 28, wherein the viral infection is an HIV infection.

31. A method of inhibiting viral replication comprising administering to a virus-infected cell an aminoglycoside antibiotic in an amount sufficient to increase the frequency of productive programmed ribosomal frameshifting events, wherein the aminoglycoside antibiotic is other than paromomycin.

32. A method of increasing the frequency of productive programmed ribosomal frameshifting events by a ribosome comprising treating a virus-infected cell with an aminoglycoside antibiotic, wherein the aminoglycoside antibiotic is administered in an amount sufficient to increase the frequency of productive programmed ribosomal frameshifting events and wherein the aminoglycoside antibiotic is other than paromoycin.

33. The method of claim 1, wherein the aminoglycoside antibiotic is capable of intereacting with a eukaryotic ribosome.

34. The method of claim 31, wherein the aminoglycoside antibiotic is gentamicin or a pharmaceutically acceptable salt thereof.
